(19) United States
(12) Patent Application Publication
(10) Pub. No.: US 2005/0196389 A1
(43) Pub. Date: Sep. 8, 2005

(54) Chlorella containing nutritional supplement having improved digestability

(76) Inventors: Nena Dockery, Springfield, MO (US); Kouji Higashida, Rancho Palos Verdes, CA (US); Tara Pearson Mooneyham, Nixa, MO (US)

Correspondence Address: Janine A. Moderson;Pauley Petersen & Erickson, Suite 365, 2800 West Higgins Road, Hoffman Estates, IL, 60195, US

(21) Appl. No.: 10/795,804

(22) Filed: Mar. 8, 2004

(51) Int. Cl. A61K 38/43; A61K 35/80

(52) U.S. Cl. 424/94.1; 424/195.17

(57) ABSTRACT

A chlorella containing nutritional supplement having improved digestibility is provided. The nutritional supplement contains chlorella and an enzyme blend including select lysing enzymes effective to lyse fibrous components of the chlorella cell wall and select microbial enzymes effective to digest carbohydrate and protein components of the chlorella. The select lysing enzymes include hemicellulase, beta-glucanase and phytase. The select microbial enzymes include bromelain, protease 4.5, protease 3.0, lipase AN, peptidase, pectinase, amylase, cellulase and glucoamylase.

### FIELD OF INVENTION

The present invention relates to a nutritional supplement containing chlorella microalgae. More particularly, the present invention relates to a nutritional supplement that contains chlorella and a blend of select enzymes effective to improve digestibility of the cellular components of the chlorella.

### BACKGROUND OF THE INVENTION

Chlorella is a microalga that emerged over 2.5 billion years ago as the first form of plant with a well-defined nucleus. Chlorella grows naturally in fresh water and has the highest content of chlorophyll of any known plant on earth. Today, chlorella is cultured on a large scale for use in nutritional supplements and foods due to its desirable biochemical profile and health promoting attributes.

For example, chlorella cells include proteins that contain all the amino acids known to be essential for the nutrition of animals and human beings. Chlorella also includes many of the vitamins and minerals that support balanced nutrition such as vitamin C, beta-carotene, lutein, thiamine, riboflavin, pyroxidine, niacin, pantothenic acid, folic acid, vitamin B-12, vitamin K, vitamin E, phosphorous, iron, calcium, potassium, magnesium, copper and zinc. In addition, chlorella contains omega fatty acids which have been found to promote cardiovascular health. Moreover, approximately 5% of each chlorella cell consists of chlorella growth factor (CGF) composed primarily of amino acids, beta-glucan and nucleic acids that are believed to be derived from the nucleus of the algae.

A number of laboratory and/or clinical studies have reported the health promoting attributes of chlorella. For example, laboratory and clinical studies have reported that disintegrated or broken chlorella cell wall, when given orally, provides nutrients that promote and enhance immunoreactivity. Furthermore, some studies have reported that the fiber and chlorophyll contained within chlorella cells are effective in the removal of toxins which promotes improved bowel health. Chlorella also displays significant antioxidant activity and provides phospholipids and other nutrients to support systemic immune and cardiovascular health as well as cognitive abilities. Thus, when detoxification, bowel cleansing, immune support and basic health maintenance are desired, chlorella preparations may be advantageously employed.

However, in its natural state, the fibrous cell wall of the chlorella cells prevents the nutrients contained within the chlorella cells from being adequately broken down, digested and absorbed. Thus, chlorella cells will generally pass through the digestive system with little or no benefit. Because the interior contents of the chlorella cells typically contain the complex mixture of nutrients that may provide most of the nutritional benefits associated with chlorella, it is generally necessary to employ some technique to break or rupture the fibrous cell wall to gain access to the cell contents.

Once access is provided to the contents of the chlorella cells enzymes must be present in order to utilize the nutrients. Generally, people absorb fewer nutrients if there is an inadequate amount of enzymes present in their digestive system. With age, there is greater likelihood that the body may produce fewer enzymes. Enzyme loss may also be attributable due to dietary deficiencies. Although the human body manufactures some enzymes, others are obtained from foods. Generally, enzymes are only found in raw foods or in those cooked at temperatures below 118° F. Typically, enzymes begin to perish when cooking temperatures rise above this level. In a diet chronically lacking in raw fruits and vegetables, it may be necessary to include enzymes in supplements that contain natural products rich in plant fiber. Enzymes generally assist in digestion and nutrient assimilation. Cellulase, for example, is an enzyme that breaks down cellulose. Although the human body produces some enzymes, it does not produce cellulase. Thus, a diet devoid of sufficient quantities of fresh, raw fruits and vegetables can contribute to digestion difficulties when cellulose-rich foods such as chlorella are introduced. As a result, fermentation and distress may follow. Thus, food enzymes are generally needed to properly digest chlorella.

In view of the above, there is a need and a demand for a chlorella containing nutritional supplement having improved digestibility. In particular, there is a need and a demand for a chlorella containing nutritional supplement that includes select enzymes that promote the break down of the fibrous components of the chlorella cell wall. There is a further need and demand for a chlorella containing nutritional supplement that includes targeted enzymes to promote effective digestion of carbohydrate and protein components contained within the chlorella cells.

### SUMMARY OF THE INVENTION

A general object of the invention is to provide a chlorella containing nutritional supplement having improved digestibility.

A more specific objective of the invention is to overcome one or more of the problems described above.

The general object of the invention can be attained, at least in part, through a nutritional supplement containing chlorella and an enzyme blend including select lysing enzymes and select microbial enzymes, wherein the enzyme blend is effective to improve digestibility of the chlorella. In accordance with certain preferred embodiments, the nutritional supplement contains about 85 to about 90 composition weight percent chlorella and about 10 to about 15 composition weight percent of the enzyme blend.

The prior art generally fails to provide a chlorella containing nutritional supplement that includes a blend of select lysing and microbial enzymes that is as effective as desired in promoting access to nutrients contained within the chlorella. Further, the prior art has generally failed to provide a chlorella containing nutritional supplement including a blend of select lysing and microbial enzymes to aid in the digestion of chlorella cell wall and cell contents to make the nutrients contained therein more readily available to the body.

The invention further comprehends a nutritional supplement containing about 88 composition weight percent chlorella and about 12 composition weight percent of an enzyme blend including select lysing and microbial enzymes that are effective to improve digestibility of the chlorella. In accordance with certain preferred embodiments, the enzyme blend advantageously includes about 2 to about 5 composition weight percent select lysing enzymes and about 95 to about 98 composition weight percent select microbial enzymes.

The invention still further comprehends a nutritional supplement containing about 85 to about 90 composition weight percent chlorella; about 0.2 to about 0.75 composition weight percent of a lysing enzyme blend including hemicellulase, beta-glucan, and phytase; and about 9.5 to about 14.7 composition weight percent of microbial enzyme blend including bromelain, protease 4.5, protease 3.0, lipase AN, peptidase, pectinase, amylase, cellulase, and glucoamylase. The nutritional supplement may be provided in a capsule or a tablet form.

Other objects and advantages will be apparent to those skilled in the art from the following detailed description taken in conjunction with appended claims.

### DESCRIPTION OF PREFERRED EMBODIMENTS

The present invention provides a chlorella containing nutritional supplement having improved digestibility. Such nutritional supplement typically includes chlorella and an enzyme blend containing select lysing enzymes and select microbial enzymes.

It is generally desirable to provide access to the contents of the chlorella cells to make the vitamins, minerals, amino acids and other nutrients available and more readily absorbable by the body. However, as discussed above, chlorella has a fibrous cell wall composed mainly of cellulose which is difficult for the human body to breakdown and digest.

Typically, chlorella is cultivated in large, fresh mineral water pools under direct sunlight. Once the fresh water pools have reached the desired density of chlorella cells, the algae are harvested and centrifuged. It is at this point that the tough cell walls of the chlorella are mechanically broken down to increase the algae's digestibility. Companies have endeavored to find the most effective way to shatter this outer shell or cell wall to release the inner contents without destroying or modifying the structural elements found within the cell wall. Each chlorella grower uses a preferred method for their species of chlorella to either crack or pulverize the cells. These typically mechanical methods include but are not limited to, jet-spraying, ultrasound treatment, heating and milling. Once the cells have been broken or cracked the chlorella is spray-dried to produce a powder or molded into tablets using a direct press machine.

While the above mechanical methods are somewhat effective for providing access to chlorella cell contents, as discussed above, the body may not be able to utilize the nutrients contained therein without the addition of enzymes typically obtained from fresh, raw fruits and vegetables. Moreover, mechanical cracking of the cell wall of the chlorella cells does not provide a mechanism to break down the fibrous components contained therein which can result in fermentation and distress in the digestive tract. Thus, it has been discovered that by combining chlorella cells ruptured by mechanical methods with select lysing enzymes and select microbial enzymes, the digestibility of the chlorella can be improved thereby making the nutrients contained within the chlorella cells more readily available to the body.

In accordance with the invention, a nutritional supplement contains chlorella and an enzyme blend including select lysing enzymes and select microbial enzymes wherein the enzyme blend is effective to improve the digestibility of the chlorella. In particular, the nutritional supplement includes lysing enzymes that are effective to lyse fibrous components of the chlorella. The nutritional supplement also includes microbial enzymes that are effective improve digestion of carbohydrate and protein components of the chlorella. As used herein, the term “effective to improve digestibility of chlorella” refers to an increase in digestibility of chlorella as measured by the AOAC pepsin digestibility test described in detail below.

In general, the nutritional supplement contains about 85 to about 90 composition weight percent chlorella. Species of chlorella suitable for use in the present invention include, but are not limited to, Chlorella vulgaris, Chlorella pyrenoidosa, and Chlorella regularis. The chlorella is desirably provided in a powdered form produced by any suitable means such as, but not limited to, spray drying broken chlorella cells. The broken chlorella cells may be obtained from any suitable mechanical cracking procedure such as, but not limited to, jet spraying, heating, ultrasound treating or milling harvested and centrifuged chlorella cells. In accordance with certain preferred embodiments, the nutritional supplement includes about 88 composition weight percent chlorella.

The nutritional supplement also contains an enzyme blend including select lysing enzymes and select microbial enzymes. Suitably, the nutritional supplement includes about 10 to about 15 composition weight percent of the enzyme blend. In accordance with certain preferred embodiments, the nutritional supplement includes about 12 composition weight percent of the enzyme blend. Desirably, the enzyme blend includes about 2 to about 5 composition weight percent select enzymes and, in certain embodiments, about 3.5 composition weight percent select lysing enzymes. Suitably, the enzyme blend includes about 95 to about 98 composition weight percent select microbial enzymes and, in certain embodiments, about 96.5 composition weight percent select microbial enzymes.

Advantageously, the lysing enzymes included in the enzyme blend are effective to lyse fibrous components of the cell wall of the chlorella cells. While various lysing enzymes may be included in the enzyme blend, select lysing enzymes contained in the nutritional supplement suitably include hemicellulase, beta-glucan and phytase. Desirably, the nutritional supplement may include about 0.2 to about 0.75 composition weight percent of the select lysing enzymes and, in certain preferred embodiments, includes about 0.4 composition weight percent of the select lysing enzymes.

Desirably, the microbial enzymes included in the enzyme blend are effective to digest carbohydrate and protein components of the chlorella cells. While various microbial enzymes may be included in the enzyme blend, suitable select microbial enzymes include bromelain, protease 4.5, protease 3.0, lipase AN, peptidase, pectinase, amylase, cellulase and glucoamylase. Advantageously, the nutritional supplement includes about 9.5 to about 14.7 composition weight percent of the select microbial enzymes and, in certain preferred embodiments, includes about 11.6 composition weight percent of the select microbial enzymes.

The nutritional supplement may be provided in any suitable form that may be readily ingested. Suitable forms include, are but not limited to, powders, capsules and tablets.

The present invention is described in further detail in connection with the following example which illustrates or simulates various aspects involved in the practice of the invention. It is to be understood that all changes that come within the spirit of the invention are desired to be protected and thus the invention is not to be construed as limited by this example.

### EXAMPLE

A nutritional supplement in accordance with the invention was prepared as follows. An enzyme blend was prepared by combining about 1.75 milligrams of a lysing enzyme blend containing hemicellulase, beta-glucanase and phytase with about 48.25 milligrams of a microbial enzyme blend containing bromelain, protease 4.5, protease 3.0, lipase AN, peptidase, pectinase, amylase, cellulase and glucoamylase. The enzyme blend was thoroughly mixed and presented in a powdered form. Thereafter, about 400 milligrams of the enzyme blend was combined with about 3000 milligrams of dried biomass of immobilized Chlorella vulgaris. The chlorella and the enzymes were blended together geometrically by weight to assure homogeneity. The combined chlorella and enzymes were further blended to a uniform and consistent product. The resulting matrix was then placed into two-piece capsules.

A comparative sample including chlorella powder from the same production lot was also prepared. The encapsulate matrix and the comparative sample were then tested in accordance with the AOAC pepsin digestibility method which is described in greater detail below.

As shown in TABLE 1 below, the encapsulated matrix including chlorella and the enzyme blend of select lysing and select microbial enzymes demonstrated improved digestibility. In particular, the encapsulated matrix of the invention showed an increase in digestibility of about 7.5%.

TABLE 1

| Sample | Pepsin-digestibility (%) |
|---|---|
| Comparative sample | 69.01 |
| Encapsulated matrix | 74.58 |

### Pepsin-Digestability Test

The pepsin-digestibility data was generated in accordance with AOAC (971.09) pepsin-digestibility test as follows.

Prior to analysis the crude or total protein content of each sample was determined according to methods known in the art. Thereafter, an approximately 1 gram sample of material to be tested was placed in a flask with a 0.2% solution of pepsin in dilute hydrochloric acid. The flask was agitated for about 16 hours in an oven set to approximately 45° C. After agitation, the contents of the flask were filtered and the undigested material collected on the filter was analyzed according to methods known in the art to determine the amount of indigestible protein present in the test sample.

Pepsin digestibility was then calculated according to the following equation:
[(Crude protein−Indigestible protein)/Crude protein]×100=% pepsin-digestibility.

While in the foregoing specification this invention has been described in relation to certain preferred embodiments thereof, and many details have been set forth for purpose of illustration, it will be apparent to those skilled in the art that the invention is susceptible to additional embodiments and that certain of the details described herein can be varied considerably without departing from the basic principles of the invention.

1. A nutritional supplement comprising:
chlorella; and
an enzyme blend including select lysing enzymes and select microbial enzymes,
wherein the enzyme blend is effective to improve digestibility of the chlorella.

2. The nutritional supplement of claim 1 wherein the select lysing enzymes are effective to lyse fibrous components of the chlorella.

3. The nutritional supplement of claim 1 wherein the select microbial enzymes are effective to improve digestion of carbohydrate and protein components of the chlorella.

4. The nutritional supplement of claim 1 wherein the select lysing enzymes comprise hemicellulase, beta-gluconase and phytase.

5. The nutritional supplement of claim 1 wherein the select microbial enzymes comprise bromelain, protease 4.5, protease 3.0, lipase AN, peptidase, pectinase, amylase, cellulase and glucoamylase.

6. The nutritional supplement of claim 1 comprising about 85 to about 90 composition weight percent chlorella.

7. The nutritional supplement of claim 1 comprising about 10 to about 15 composition weight percent of the enzyme blend.

8. The nutritional supplement of claim 1 wherein the enzyme blend comprises about 2 to about 5 composition weight percent of select lysing enzymes.

9. The nutritional supplement of claim 1 wherein the enzyme blend comprises about 95 to about 98 composition weight percent of select microbial enzymes.

10. A nutritional supplement comprising:
about 88 composition weight percent chlorella; and
about 12 composition weight percent of an enzyme blend including select lysing enzymes and select microbial enzymes,
wherein the enzyme blend is effective to improve digestibility of the chlorella.

11. The nutritional composition of claim 10 wherein the select lysing enzymes are effective to lyse fibrous components of the chlorella.

12. The nutritional supplement of claim 10 wherein the select microbial enzymes are effective to digest carbohydrate and protein components of the chlorella.

13. The nutritional supplement of claim 10 wherein the select lysing enzymes comprise hemicellulase, beta-gluconase and phytase.

14. The nutritional supplement of claim 10 wherein the select microbial enzymes comprise bromelain, protease 4.5, protease 3.0, lipase AN, peptidase, pectinase, amylase, cellulase and glucoamylase.

15. The nutritional composition of claim 10 wherein the enzyme blend comprises about 3.5 composition weight percent of select lysing enzymes.

16. The nutritional composition of claim 10 wherein the enzyme blend comprises about 96.5 composition weight percent of select microbial enzymes.

17. A nutritional supplement comprising:
about 85 to about 90 composition weight percent chlorella;
about 0.2 to about 0.75 composition weight percent of a lysing enzyme blend including hemicellulase, beta-glucanase, and phytase; and
about 9.5 to about 14.7 composition weight percent of a microbial enzyme blend including bromelain, protease 4.5, protease 3.0, lipase AN, peptidase, pectinase, amylase, cellulase and glucoamylase.

18. The nutritional supplement of claim 17 comprising:
about 88 composition weight percent chlorella;
about 0.4 composition weight percent of the lysing enzyme blend; and
about 11.6 composition weight percent of the microbial enzyme blend.

19. The nutritional supplement of claim 17 provided in a capsule.

20. The nutritional supplement of claim 17 provided in a tablet.
